(19) United States
(12) Patent Application Publication
(10) Pub. No.: US 2026/0258489 A1
(43) Pub. Date: Sep. 3, 2026

(54) METHODS AND KITS FOR INTRACELLULAR AMPLIFICATION AND SEQUENCING (FLUORESCENTLY LABELED INTRACELLULAR GENOMICS (FLING))

(71) Applicants: Kerry Geiler-Samerotte, Scottsdale, AZ (US); Leandra Brettner, Mesa, AZ (US); Parker Crossland, Phoenix, AZ (US); Kara Schmidlin, Mesa, AZ (US)

(72) Inventors: Kerry Geiler-Samerotte, Scottsdale, AZ (US); Leandra Brettner, Mesa, AZ (US); Parker Crossland, Phoenix, AZ (US); Kara Schmidlin, Mesa, AZ (US)

(73) Assignee: ARIZONA BOARD OF REGENTS ON BEHALF OF ARIZONA STATE UNIVERSITY, Scottsdale, AZ (US)

(21) Appl. No.: 19/553,177

(22) Filed: Feb. 27, 2026

Related U.S. Application Data
(60) Provisional application No. 63/765,348, filed on Feb. 28, 2025.

(51) Int. Cl. C12Q 1/6869; C12Q 1/6841; G01N 1/30

(52) U.S. Cl. CPC C12Q 1/6869; C12Q 1/6841; G01N 1/30; G01N 2001/305

(57) ABSTRACT

Rare genotypes in bacterial or eukaryotic populations are essential for understanding adaptation, ecology, and disease, but assembling the full genome of rare cell types remains challenging. Bulk sequencing of mixed populations obscures rare genotypes and haplotype information, while random single-cell isolation is labor-intensive, particularly for rare cell types. Described here is a novel method that enriches cells with a known genetic marker, enabling efficient sequencing of their partial or full genome. This approach integrates intracellular genomic DNA amplification, Fluorescent In Situ Amplification (FISA), Fluorescence-Activated Cell Sorting (FACS), and whole-genome sequencing. It allows robust amplification and sequencing of single genes and entire genomes from formaldehyde-fixed cells, preserving cellular integrity and minimizing false positives. This versatile method provides a powerful tool for studying rare genotypes and genetic heterogeneity across applications in evolution, ecology, and medicine.

### CROSS-REFERENCE TO RELATED APPLICATIONS

This application is a non-provisional and claims benefit of U.S. Provisional Application No. 63/765,348 filed Feb. 28, 2025, the specification of which is incorporated herein in its entirety by reference.

This application also incorporates by reference U.S. application Ser. No. 19/553,141 filed Feb. 27, 2026 in its entirety.

### STATEMENT REGARDING FEDERALLY SPONSORED RESEARCH OR DEVELOPMENT

This invention was made with government support under Grant No. R35 GM133674 awarded by National Institutes of Health and Grant No. 2119963 awarded by National Science Foundation. The government has certain rights in the invention.

### FIELD OF THE INVENTION

The present invention provides methods for detecting, isolating, and sequencing rare cell types based on genetic variations at a single locus, enabling robust amplification of genes and genomes from fixed cells while preserving integrity and minimizing false positives. The invention also includes related kits.

### BACKGROUND OF THE INVENTION

Despite major advances in genome sequencing technologies, it remains difficult to understand genetic diversity in mixed cell populations because common genotypes are sequenced much more often than rare ones. Detecting the presence of rare genotypes in microbial or other mixed cell populations and subsequently sequencing the genomes of those rare cells would provide transformative insights across numerous biological disciplines. For instance, microbial ecologists aim to decipher which species comprise microbiomes, how each contributes to the metabolome, as well as the roles of specific genes in microbe-host, microbe-microbe, and microbe-environment interactions. In many cases, bulk sequencing has revealed the presence of either rare or environmentally impactful genotypes in the population, such as those related to nitrogen fixation or drug resistance. Tools to tease out, sequence and study rare cells carrying specific genes could improve our ability to understand and predict the causal effects of the microbiome on the metabolome, clarify the role of horizontal gene transfer in the evolution of drug resistance and uncover missing branches in the tree of life, advancing our understanding of microbial diversity. Beyond microbiome research, evolutionary biologists conducting laboratory evolution experiments also struggle to detect rare adaptive mutations or haplotypes. They rely either on bulk DNA sequencing of entire evolved populations—where signals from rare genotypes are often drowned out by the majority—or on isolating cells one at a time—an approach limited by randomness and labor intensity. Similarly, an inability to sequence rare genomes impacts the field of medicine, where pathogenic or drug-resistant isolates do not exist in clonal populations, but are heterogeneous in that multiple different adaptive genotypes co-exist in clinical samples. The ability to resolve the genetic types present within these mixed populations is badly needed and could help correctly diagnose and improve treatment outcomes for patients presenting with meningitis, encephalitis, pneumonia, sepsis, and other infectious diseases.

### BRIEF SUMMARY OF THE INVENTION

It is an objective of the present invention to provide methods and kits that allow for intracellular amplification of DNA within fixed cells while preserving the ability to later sequence the DNA from those cells, as specified in the independent claims. Embodiments of the invention are given in the dependent claims. Embodiments of the present invention can be freely combined with each other if they are not mutually exclusive.

The present invention addresses the aforementioned challenges involved in studying mixed cell populations by presenting a novel platform called Fluorescently Labeled INtracellular Genomics (FLING) (FIG. 1A-1E). This method enables the detection, isolation, and sequencing of rare cell types of interest based on known genetic differences at a single locus. FLING begins with the fixation and permeabilization of live cells (FIG. 1A), allowing the amplification reagent to diffuse into the intracellular space. Next, intracellular DNA amplification is performed using an isothermal polymerase and (optionally biotinylated, or otherwise modified) primers targeting a genetic sequence present in the rare cell type of interest, with EdU (5-ethynyl-2′-deoxyuridine) incorporated into newly synthesized DNA at thymine sites (FIG. 1B). This process, termed Fluorescent In Situ Amplification (FISA), fluorescently labels amplified DNA via a click chemistry reaction (e.g., Click-iT® reaction), enabling detection by flow cytometry (FIG. 1C). Flow cytometry identifies cells with fluorescence signals above a threshold, which distinguishes cells containing amplified DNA from controls where no amplification occurred. The shift in fluorescence distribution allows rare genotypes of interest to be identified and enriched. Fluorescent cells are then sorted based on their fluorescence intensity (FIG. 1D) and can undergo further DNA amplification, including additional rounds of intracellular DNA amplification to improve recovery of full genomes, and/or post-lysis whole genome amplification (WGA) using multi-strand displacement amplification (MDA). Finally, DNA from sorted cells is collected, optionally using streptavidin beads to bind and enrich for intracellularly amplified DNA, prepared for sequencing, and run on a sequencing machine to enable identification of single nucleotide polymorphisms or other genetic features of interest in the rare genotype of interest (FIG. 1E).

The FLING approach offers several advantages over current methodologies. Unlike bulk sequencing, which obscures rare genotypes, the presently claimed method can isolate specific genotypes and even individual cells of interest, allowing detailed investigation of haplotype structures and phase information (e.g., which reads come from the same genotype or cell). And unlike most other methods to isolate rare genotypes of interest, FLING does not require randomly choosing and isolating single cells in the hopes of selecting the genotype of interest because it enriches for genotypes of interest and allows high-quality whole genome information to be recovered from cells possessing those genotypes. FLING also avoids cell lysis prior to the intracellular DNA amplification steps, which enables multiple rounds of washing away reagents, potentially facilitating subsequent amplification reactions of multiple targets within the same cells (e.g. DNA+RNA targets). FLING improves upon methods such as those that enrich genotypes of interest by using fluorescent in situ hybridization to tag cells of interest based on their RNA because (unlike RNA methods) FLING does not require expression of the genomic region that identifies the cells of interest. This enables the application of FLING to isolate and sequence a broader range of cell types, including barcoded strains of interest from engineered libraries, cancer cells with mutations of interest to lowly expressed genes, and bacterial cells that possess but do not express drug-resistant cassettes in their current environments.

Earlier efforts at performing amplification of genetic material within fixed cells have encountered significant limitations. For example, in situ PCR often damages cell membranes due to the high temperatures required for DNA denaturation and primer annealing, leading to extracellular amplification and false positives or negatives. Previous work also often maintains cells on slides or embedded within parafilm, which may help contend with cell damage caused by temperature and harsh fixatives, but this limits subsequent studies such as those that require separating out the cells of interest.

Perhaps the most significant and major limitation of previous work using in situ amplification and/or FISH has been the difficulty of sequencing genomic DNA from formaldehyde-fixed cells. Thus, while previous in situ PCR techniques have proven useful in allowing researchers to identify the presence of, or track changes in the frequencies of, genotypes of interest within mixed populations, investigating the full genomes of rare and interesting cells remains elusive. More generally speaking, many previous studies discuss how sequencing the genomes of formaldehyde fixed cells was, with existing technology, impractical (Clingenpeel S, Schwientek P, Hugenholtz P, Woyke T (2014) Effects of sample treatments on genome recovery via single-cell genomics. ISME J 8:2546-2549; Pereira A C, Tenreiro A, Cunha M V (2022) When FLOW-FISH met FACS: Combining multiparametric, dynamic approaches for microbial single-cell research in the total environment. Sci Total Environ 806:150682; Kirsch J M, Hryckowian A J, Duerkop B A (2024) A metagenomics pipeline reveals insertion sequence-driven evolution of the microbiota. Cell Host Microbe 32:739-754.e). The difficulty is generally ascribed to the formaldehyde used during cell fixation. Formaldehyde crosslinks stabilize genetic material inside the cell for hybridization or amplification, but these same crosslinks have been blamed for impeding whole-genome amplification and downstream sequencing. While some studies have successfully amplified and sequenced very short, engineered DNA sequences (15 bp) or RNA transcripts, which are inherently limited in length, sequencing whole genomes from formaldehyde-fixed tissue samples or cells has, until now, proven elusive. This limitation has driven efforts to perform FISH and FACS in live cells, with little success (Pereira A C, Tenreiro A, Cunha M V (2022) When FLOW-FISH met FACS: Combining multiparametric, dynamic approaches for microbial single-cell research in the total environment. Sci Total Environ 806:150682). Given the revolutionary insights enabled by whole genome sequencing, the ability to sequence full genomes of rare cell types after they are identified via FISA or FISH would unlock vast new applications.

In some embodiments, the present invention features a method for detecting, isolating, and most importantly sequencing a genotype of interest from a mixed population of cells. In some embodiments, the method comprises a) fixating and permeabilizing a liquid sample comprising the mixed population of cells, b) amplifying a genetic marker that is unique to the genotype of interest such as an insertion, single nucleotide polymorphism, or engineered barcode within the fixed and permeabilized cells by incubating the cells a solution comprising at least an isothermal polymerase, c) generating a fluorescent signal within the cells comprising the genotype of interest, d) detecting and isolating the fluorescent cells, and e) performing subsequent reactions on the isolated cells including further intracellular reactions such as DNA amplification and/or reverse transcription, and/further post cell lysis reactions such as MDA or WGS.

In some embodiments, the present invention may also feature a kit for detecting, isolating, and sequencing a genotype of interest from a mixed population of cells. In some embodiments, the kit comprises a fixative agent, permeabilization agent, one or more nucleic acid primers, an isothermal polymerase, and nucleotide analogue (e.g., 5-ethynyl-2′-deoxyuridine (EdU)). In other embodiments, the kit comprises a fixative agent, permeabilization agent, one or more nucleic acid primers, an isothermal polymerase, and fluorescent DNA probe.

One of the unique and inventive technical features of the present invention is the use of an isothermal polymerase, biotinylated primers and EdUs (5-ethynyl-2′-deoxyuridines). Without wishing to limit the invention to any theory or mechanism, it is believed that these technical features of the present invention advantageously allow for amplifying DNA within a fixed cell (isothermal polymerase), enriching for cells with amplified DNA (EdUs) and enriching and purifying amplified DNA post lysis (biotinylated primers).

Also, without wishing to limit the invention to any theory or mechanism, it is believed that the technical features of the present invention advantageously allow for intracellular amplification of nucleic acids. This is counter to the traditional strategy of first isolating nucleic acids from a cell (for example, by lysing the cell), and only then (after isolation) amplifying said nucleic acids.

Further, without wishing to limit the invention to any theory or mechanism, it is believed that the technical features of the present invention advantageously allow for selective amplification of genetic material belonging to only a subset of cells (selected for amplification because they possess a genotype of interest) among a larger population of cells, some of which lack the genotype of interest. This includes (but is not limited to), for example, separating out cells with a genome of interest that exhibit fluorescence, or a degree of fluorescence, that distinguishes them from cells lacking the genotype of interest.

Additionally (or alternatively), without wishing to limit the invention to any theory or mechanism, it is believed that the present invention is also capable of achieving amplification of all genetic material within a population of cells, but then surprisingly facilitating isolation of only those cells containing a genome of interest. For example, using the techniques of the prior invention, it is possible to amplify the genetic material of all cells in a population (including those without the genome of interest), then select only those cells that contain the genome of interest, and separate those cells from the rest of the cells in the population. None of the presently known prior references or works have the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A-1E show Fluorescently Labeled INtracellular Genomics (FLING) combines Fluorescent in situ Amplification (FISA) with Cell Sorting and Sequencing. This figure depicts the steps involved in FLING (Fluorescently Labeled INtracellular Genomics). FIG. 1A shows the fixation and permeabilization of live cells to allow diffusion of amplification reagents into the intracellular space. FIG. 1B shows intracellular DNA amplification using a thermostable polymerase with EdU (5-ethynyl-2′-deoxyuridine) incorporation into newly synthesized DNA, followed by the click chemistry (e.g., Click-iT®) reaction to fluorescently label EdU-incorporated DNA. This portion of the FLING method is called FISA (Fluorescent In Situ Amplification). FISA can be done with random hexamers that amplify the entire genome, but to identify rare cells of interest in a mixed population it is more typically done with specific primers that target a DNA sequence that is only present in that rare cell type. FIG. 1C shows flow cytometry to detect fluorescence signals, thereby identifying cells possessing newly amplified DNA, and in so doing, identifying which cells possess the gene of interest. Toy histograms depict the distributions of fluorescence signals in a control cell population where no DNA amplification occurred and a test cell population where many cells underwent FISA. Depending on the rarity of the target genotype, the mode of this test distribution and/or its skewness will shift (see examples in subsequent figures). FIG. 1D shows sorting of cells above a threshold level of fluorescence defined by the difference between the test and control distributions to isolate fluorescently labeled cells for downstream analysis. FIG. 1E shows sequencing of sorted fluorescent cells to recover genomic data from these rare cell types of interest, enabling applications such as mapping mutations, resolving haplotypes, and studying the genomes of unculturable microbes or mixed populations. Optionally, prior to lysis of the sorted cells, a second round of FISA can be performed on the sorted cells using biotinylated random hexamers. This both increases the amount of genomic DNA available for sequencing and enables this DNA to be bound to streptavidin beads and washed free of any contaminants. After cell lysis and optionally bead-binding (if biotinylated primers were used) and washing intracellularly amplified DNA, any recovered DNA can be further amplified and prepared for sequencing.

FIG. 2 shows the challenge that is resolved by FLING, which is that rare genotypes that exist within a mixed population are impossible to sequence. In the left panel, two rare genotypes each present in less than 1% of cells exist in a mixed yeast population. These genotypes are interesting because while they resist single drugs they are sensitive to the drug combination. The right panel depicts how isolating these two genotypes would require randomly hand picking and sequencing thousands of single colonies until the desired genotype happens to be selected, based on a binomial distribution. Instead, FLING can enrich for each genotype of interest in just one experiment.

FIG. 3 shows FISA can be used in prokaryotes and eukaryotes to intracellularly amplify DNA.

FIG. 4 shows FISA can be used to intracellularly amplify and subsequently sequence the entire genomes of formaldehyde fixed yeast and bacterial cells.

FIG. 5 shows the recovery of long pieces of DNA consistent with the use of a polymerase that makes long pieces of DNA.

### DETAILED DESCRIPTION OF THE INVENTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessarily solely”. Furthermore, variations of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

As used herein, “fixation” or “fixing” refers to the process of chemically stabilizing organic, inorganic, or a combination of organic and inorganic molecules through the use of reagents, known as “fixatives”. Exemplary fixatives for the present disclosure include, but are not limited to, formaldehyde, formaldehyde derived from paraformaldehyde, formalin, phosphate buffered formalin, formal calcium, formal saline, zinc formalin, alcoholic formalin, glutaraldehyde, other organic aldehydes, methanol, ethanol, isopropanol, or other organic alcohols, or solutions containing organic alcohols or aldehydes.

As used herein, “permeabilization” or “permeabilizing” refers to the process of introducing openings into barriers to allow the penetration of desired molecules past the aforementioned barrier. In some embodiments, the barrier comprises a cell membrane, and or a cell wall. In some embodiments, permeabilization is performed by, for example, enzymes on biological membranes. Exemplary enzymes for permeabilization of biological membranes include, but are not limited to, lysozyme, proteinase K, and zymolyase. In some embodiments, permeabilization is performed by, for example, detergents on biological membranes such as Triton or Tween.

As used herein, “amplification” refers to the process of replicating nucleic acid strands by enzyme-catalyzed extension. Exemplary enzymes for amplification of nucleic acids in the current disclosure include, for example, nucleic acid polymerases. In some embodiments, an isothermal polymerase is used to amplify nucleic acids. In some embodiments, amplification is carried out with a high-fidelity polymerase, such as Q5, with the technique known as the polymerase chain reaction (PCR). Amplification can be performed with natural and non-natural nucleotide bases, ribonucleotide bases or deoxyribonucleotide bases, labeled nucleotide bases, and the like.

As used herein, “isothermal amplification” describes amplification of DNA targets without heat denaturation of DNA. In contrast, polymerase chain reaction (PCR) requires cycling through different temperatures for denaturation, hybridization, and extension. Isothermal amplification may be preceded by a higher temperature hybridization step that does not denature the DNA target. Exemplary polymerases useful for isothermal amplification are referred to herein as isothermal polymerases, and include, but are not limited to phi29 polymerase, Klenow exo-DNA Polymerase I, Bsu polymerase, Bst polymerase, Bsm polymerase.

As used herein, “sequencing” refers to the sequencing of nucleic acids. Sequencing of nucleic acids may be accomplished using, by way of example but not by way of limitation, PacBio long read sequencing, Sanger sequencing, or next-generation sequencing.

As used herein, the term “random hexamer” or “random hexonucleotide” refers to a region of six nucleotides in length comprising sequences that are synthesized at random. The purpose of random hexamers is, in most applications, to bind complementarily to nucleotide sequences of unknown identity. Thus, because random hexamers theoretically cover all possible sequence permutations for a hexameric (6-member) nucleotide, they are likely to bind at many positions to nucleotides of any sequence. It should be understood, however, that a key feature of random hexamers is not that they are six nucleotides in length, but rather that they have random sequence identity. In other words, for many applications it is possible to provide random pentamers (5-member), heptamers (7-member), or other random sequences in place of hexamers. In some embodiments, a random hexamer comprises a part of, or a portion of a larger oligonucleotide, such as an oligonucleotide primer.

As used herein, “crowding agent” refers to compounds that decrease the solvent available to macromolecules, thereby increasing the relative concentration of said macromolecules and altering their properties. In some applications, crowding agents have the effect of increasing enzyme activity and accelerating reactions resulting in faster and potentially more specific assays. In some embodiments, crowding agents may include one or more of polyethylene glycol (PEG), polyethylene glycol 8000 (PEG-8000), trehalose, and sorbitol. In some embodiments crowding agents may include ficoll or dextrans.

Rare genotypes within bacterial or eukaryotic cell populations are critical for understanding adaptation, ecological dynamics, and disease, but it is difficult to assemble the full genome of a rare cell type with current methods. Bulk sequencing of mixed microbial populations not only can obscure rare genotypes, but also does not preserve haplotype information (in other words, it is not always clear which DNA sequences belong to which genome). On the other hand, single-cell isolation, which avoids the problems characteristic of bulk sequencing, is highly labor-intensive and becomes increasingly so the rarer the cell-type of interest. Here, the present invention features a novel method (known as FLING for Fluorescently Labeled INtracellular Genomics) that enriches for cells with a known genetic marker of interest allowing the full genome of cells possessing that marker to be easily sequenced. The FLING method described herein combines intracellular genomic DNA amplification with Fluorescent In Situ Amplification (FISA), Fluorescence-Activated Cell Sorting (FACS), and whole-genome sequencing. This method enables robust amplification and sequencing of single genes and entire genomes from formaldehyde-fixed cells while preserving cellular integrity and minimizing false positives. This versatile method offers a powerful tool for studying rare genotypes and genetic heterogeneity in contexts ranging from evolution to medicine.

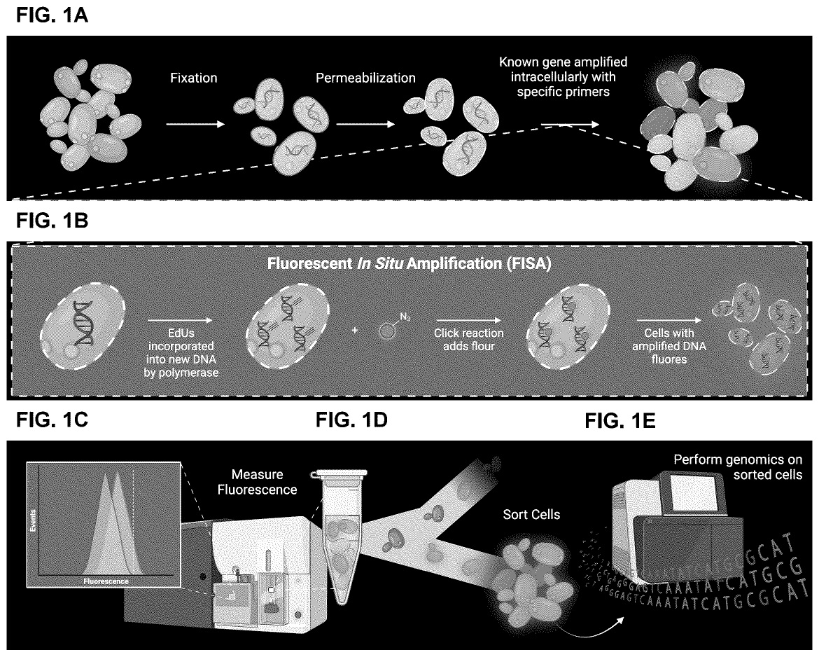

Referring now to FIGS. 1A-5, the present invention features a novel method (i.e., Fluorescently Labeled INtracellular Genomics (FLING)) that enriches cells with a known genetic marker of interest allowing the full genome of cells possessing that marker to be easily sequenced. The method described herein enables the detection, isolation, and sequencing of rare cell types of interest based on known genetic differences at a single locus. The FLING method combines intracellular genomic DNA amplification with Fluorescent In Situ Amplification (FISA), Fluorescence-Activated Cell Sorting (FACS), and whole-genome sequencing. This method enables robust amplification and sequencing of single genes and entire genomes from formaldehyde-fixed cells while preserving cellular integrity and minimizing false positives. This method isolates individual cells of interest, allowing detailed investigation of haplotype structures and phase information. Also, FLING does not require labor-intensive random isolation of cells and avoids cell lysis prior to, at least some, DNA amplification steps. This versatile method offers a powerful tool for studying rare genotypes and genetic heterogeneity in contexts ranging from evolution to medicine.

A method for detecting, isolating, and sequencing a genotype of interest from a mixed population of cells, the method comprising: fixating and permeabilizing a sample comprising the mixed population of cells; amplifying the genotype of interest within the fixed and permeabilized cells by incubating the cells in a solution comprising at least an isothermal polymerase; generating a fluorescent signal within the cells comprising the genotype of interest; detecting and isolating the fluorescent cells; and sequencing the isolated cells.

In some embodiments, the present invention features a method for detecting, isolating, and sequencing a genotype of interest from a mixed population of cells. In some embodiments, the method comprises a) fixing and permeabilizing a liquid sample comprising the mixed population of cells, b) amplifying the genotype of interest within the fixed and permeabilized cells by incubating the cells in a solution comprising at least an isothermal polymerase, c) generating a fluorescent signal within the cells comprising the genotype of interest, d) detecting and isolating the fluorescent cells, and e) sequencing the isolated cells.

In other embodiments, the method comprises a) fixing and permeabilizing a liquid sample comprising the mixed population of cells, b) amplifying the genotype of interest within the fixed and permeabilized cells by incubating the cells in a solution comprising at least an isothermal polymerase and a nucleotide analogue (e.g., EdU), c) incubating the cells with an azide to generate a fluorescent signal within the cells comprising the genotype of interest, d) detecting and isolating the fluorescent cells, and e) sequencing the isolated cells. The present invention is not limited to incubation solely with an azide to generate a fluorescent signal. It may also include other solutions capable of activating and inducing fluorescence in any nucleotide analogue incorporated into the newly amplified DNA.

In certain embodiments, the method comprises a) fixing and permeabilizing a liquid sample comprising the mixed population of cells, b) amplifying the genotype of interest within the fixed and permeabilized cells by incubating the cells in a solution comprising at least an isothermal polymerase, c) incubating the cells with a fluorescently labeled DNA probe to generate a fluorescent signal within the cells comprising the genotype of interest, d) detecting and isolating the fluorescent cells, and e) sequencing the isolated cells. In some embodiments, the DNA probe has sequence homology to the genotype of interest.

In some embodiments, the present invention features a method for detecting, isolating, and sequencing a genotype of interest from a mixed population of cells. In some embodiments, the method comprises a) fixing and permeabilizing a liquid sample comprising the mixed population of cells, b) amplifying the genotype of interest within the fixed and permeabilized cells by incubating the cells in a solution comprising at least an isothermal polymerase, c) generating a fluorescent signal within the cells comprising the genotype of interest, d) detecting and isolating the fluorescent cells, and e) performing another round of intracellular reactions on the sorted cells such as amplifying their whole genomes by incubating the cells with a solution containing at least an isothermal polymerase and biotinylated random hexamers, f) lysing the cells, binding biotin labeled DNA to streptavidin beads, reamplifying and sequencing this DNA.

In some embodiments, the present invention features a method for detecting, isolating, and sequencing a genotype of interest from a mixed population of cells. In some embodiments, the method comprises a) fixing and permeabilizing a liquid sample comprising the mixed population of cells, b) amplifying the genotype of interest within the fixed and permeabilized cells by incubating the cells in a solution comprising at least an isothermal polymerase, c) generating a fluorescent signal within the cells comprising the genotype of interest, d) detecting and isolating the fluorescent cells, and e) performing another round of intracellular reactions on the sorted cells such as reverse transcribing and labeling their RNA transcripts with barcodes and biotin, f) lysing the cells and sequencing their DNA and RNA.

In some embodiments, isothermal polymerase comprises a phi29 polymerase. In other embodiments, the isothermal polymerase is a Bst polymerase. Without wishing to limit the present invention to any theory or mechanism it is believed that the use of an isothermal polymerase is advantageous in that it (1) amplifies DNA at low temperatures that do not damage cell membranes and (2) can contend with formaldehyde crosslinks via its strand displacing ability.

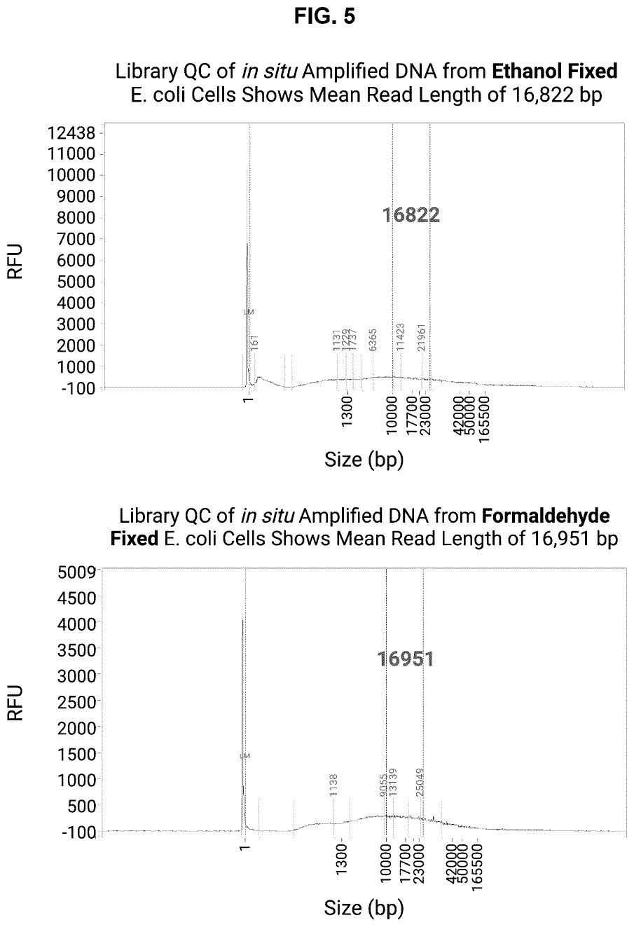

In some embodiments, the methods described herein allow for successful intracellular amplification and sequencing of target genes and/or the whole genomes of species with different types of cell membranes including fungal cells (e.g., S. cerevisiae), gram-positive bacteria (e.g., B. subtilis), and gram-negative bacteria (e.g., E. coli). In some embodiments, formaldehyde crosslinks do not disrupt the isothermal polymerase as demonstrated by figures showing that the amplified pieces of DNA are very long (up to 100 KB) (see for example FIG. 5).

Additionally, without wishing to limit the present invention to any theory or mechanism it is believed that the methods described herein contend with the challenge of amplifying DNA post cell lysis. Lysis buffers can be harsh and interfere with DNA amplification. Formaldehyde may exacerbate this issue by requiring stronger lysis buffers. But since DNA amplification can be performed intracellularly (before cell lysis) with biotinylated primers, intensive chemicals can be used to lyse cells post amplification, as they will no longer interfere. This is because the use of biotinylated primers (see below) allows the lysis buffer to be washed away while biotinylated, intracellularly-amplified DNA is bound to streptavidin beads. This facilitates subsequent amplification and DNA sequencing reactions using the bead-bound DNA as template.

In some embodiments, the solution used to incubate cells to facilitate intracellular amplification further comprises enzymes, dNTPs, a buffer, and one or more nucleic acid primers. As referred to herein, “nucleic acid primers” may include, but are not limited to, modified nucleic acid primers, e.g., biotinylated nucleic acid primers.

In other embodiments, the solution further comprises a nucleotide analogue, enzymes, dNTPs, a buffer, and one or more nucleic acid primers. In some embodiments, the nucleotide analogue comprises 5-ethynyl-2′-deoxyuridine (EdU). The present invention is not limited to the aforementioned nucleotide analogues and may also encompass other biomolecules, such as fluorescent dNTPs, which can be incorporated into newly amplified DNA to confer fluorescence.

In some embodiments, the nucleic acid primers comprise modified nucleic acid primers. In some embodiments, the nucleic acid primers comprise biotinylated primers. In some embodiments, the nucleic acid primers comprise sequence-specific primers. In certain embodiments, the nucleic acid primers include random oligonucleotide primers. For example, random oligonucleotide primers (e.g., random hexamers) may be used, and the amplification process can be repeated multiple times on the same cell (e.g., a fixed and permeabilized cell) with different sets of primers. In such embodiments, the genotype of interest may first be amplified using sequence-specific primers, followed by amplification with random hexamer primers.

In some embodiments, the fluorescent cells are separated by flow cytometry, microscopy, or microfluidics. In some embodiments, separating the fluorescent cells comprises separating single fluorescent cells each into independent wells or containers. In other embodiments, the fluorescent cells are separated from the less fluorescent cells into a pool by flow cytometry, microscopy, or microfluidics rather than into individual wells.

In some embodiments, the aforementioned methods further comprise lysing the separated cells. In other embodiments, the aforementioned methods further comprise lysing the separated cells and amplifying DNA from said cells. In some embodiments, amplifying the DNA in the separated cells comprises introducing a second solution comprising enzymes, dNTPs, a buffer, and one or more nucleic acid primers either pre or post lysis.

Biotinylation is one of several optional implementations of the present technology that allow for more diverse downstream applications. In one implementation, the primers used to intracellularly amplify DNA are biotinylated, or labeled with another molecule that allows capture after cell lysis. This allows intracellularly amplified DNA to be bound to streptavidin beads after cell lysis (because biotin binds streptavidin). Any unbound DNA, which represents native genomic DNA or contaminants, can then be washed away. This implementation could be useful when there is only a small amount of template DNA (i.e. single-cell sequencing) because in that case the template is more likely to be overwhelmed by contaminants. This method of intracellularly amplifying DNA with biotinylated primers allows such contaminants to be washed away leaving only the intracellularly amplified pieces of interest.

Another optional implementation is the use of Click chemistry (e.g., “Click-It®”) to make the intracellularly amplified DNA fluoresce. In this implementation, EDUs, which are typically fed to growing cells, are instead diffused into dead, fixed cells. Here, they are incorporated into intracellularly amplified DNA. Incubation of these cells with an azide-bound Alexa Fluor™ results in the cells with intracellularly-amplified DNA giving off a fluorescent signal. This approach of detecting which cells possess intracellularly-amplified DNA is superior to one that involves a probe because in this method, only the intracellularly amplified DNA gives off a fluorescent signal. This can help distinguish cells with amplified DNA from other cells.

In sum, the present invention provides a proven method to intracellularly amplify DNA that allows for downstream DNA sequencing. This ability is ripe for other, optional downstream applications including reactions involving barcoding, single-cell sequencing, sorting, rare cell genomics, etc.

### Kits for Detecting, Isolating, and Sequencing a Genotype of Interest

In some embodiments, the present invention may also feature a kit for detecting, isolating, and sequencing a genotype of interest from a mixed population of cells. In some embodiments, the kit comprises a fixative agent, permeabilization agent, one or more nucleic acid primers, an isothermal polymerase, and a nucleotide analogue (e.g., 5-ethynyl-2′-deoxyuridine (EdU)). In other embodiments, the kit comprises a fixative agent, permeabilization agent, one or more nucleic acid primers, an isothermal polymerase, and fluorescent DNA probe. In other embodiments the kit comprises: one or more fixative agent; one or more permeabilization agent; an isothermal polymerase; and a nucleotide analogue; and an azide bound fluorophore configured to bind the nucleotide analogue. In other embodiments, the kit comprises: one or more fixative agent; one or more permeabilization agent; one or more nucleic acid primers; an isothermal polymerase; and a fluorescent DNA probe. The nucleotide analogues selected may be non-fluorescent or fluorescent, unless otherwise indicated.

In some embodiments, the kit is a unified assembly of reagents provided in one or more containers, configured to perform the methods described herein.

In some embodiments, fixative agents are used to preserve the structural integrity of cells. Fixative agents may include aldehydes (e.g., formaldehyde, glutaraldehyde), alcohols (e.g., ethanol, methanol), or metallic salts. In some embodiments, the fixative agent(s) comprise formaldehyde and/or ethanol.

In some embodiments, permeabilization agents are configured to increase the porosity of cellular membranes to allow entry of macromolecules. Permeabilization agents include, but are not limited to, detergents (e.g., Triton™ X-100, NP-40, Saponin) or organic solvents. In some embodiments, the permeabilization agent(s) comprises an enzymatic treatment and/or a detergent. In some embodiments, the enzymatic treatment comprises zymolyase. In some embodiments, the detergent comprises Tween-20 or Triton X-100.

In some embodiments, nucleic acid primers are sets of oligonucleotides specifically designed to hybridize with a target sequence. These may be random hexamers, oligo (dT) primers, or sequence-specific primers. In some embodiments, the nucleic acid primers comprise biotinylated primers, other modified nucleic acid primers, sequence-specific primers, or random oligonucleotide primers.

In some embodiments, isothermal polymerases are strand-displacing polymerase capable of nucleic acid synthesis at a constant temperature. In some embodiments, the isothermal polymerase comprises phi29 polymerase or Bst polymerase.

In some embodiments, nucleotide analogues (e.g., 5-ethynyl-2′-deoxyuridine (EdU)) are used for incorporation into de novo synthesized DNA during nucleic acid replication or amplification.

In some embodiments, the fixative agents and permeabilization agents are configured to ensure the nucleotide analogues (e.g., 5-ethynyl-2′-deoxyuridine (EdU)) and nucleic acid primers can access the intracellular environment, and the isothermal polymerase is selected for its strand-displacing ability which facilitates sequencing of DNA that has undergone formaldehyde cross-linking.

In some embodiments, the kit further comprises reaction buffers (“buffers”).

Buffers may contain magnesium ions, salts (e.g., NaCl, KCl, etc.), and stabilizing agents (e.g., Bovine Serum Albumin (BSA), sorbitol, etc.). In some embodiments, the buffers may be configured for the specific isothermal polymerase selected.

In some embodiments, the kit further comprises a detection reagent, e.g., a click-chemistry detection reagent, including, but not limited to, a fluorescent probe, including a fluorescent azide (e.g., Alexa Fluor™).

In some embodiments, the kits of the present invention are configured for a “one-pot” reaction. In some embodiments, the kits of the present invention are configured for sequential reactions. In some embodiments, the kit may comprise further instructions for detecting, isolating, and sequencing a genotype of interest from a mixed population of cells.

In some embodiments, methods (e.g., instructions) of using the kit for detecting, isolating, and sequencing a genotype of interest from a mixed population of cells comprise the following steps:
Step 1: Fixation and Permeabilization of Cells. In some embodiments, a biological sample (e.g., adherent cells, tissue sections, or cell suspensions) is first contacted with the fixative agent for a period of 10 to 30 minutes at room temperature, which preserves cellular morphology. In some embodiments, following fixation, the biological sample is treated with the permeabilization agent. Use of the permeabilization agent creates pores in the lipid bilayer of the cells in the sample, ensuring nucleic acid primers, isothermal polymerase, and other reaction reagents can enter the cell membrane to reach the intracellular target nucleic acids to be amplified.
Step 2: Nucleic Acid Priming, EdU Incorporation. In some embodiments, the permeabilized sample of cells is then incubated with a reaction mixture containing nucleic acid primers, isothermal polymerase, and detection reagent (e.g., fluorescent probe, e.g., 5-ethynyl-2′-deoxyuridine (EdU)). Because an isothermal polymerase is utilized, the reaction mixture does not need to be thermal cycled, and may be kept at a constant temperature, depending on the specific isothermal polymerase used (e.g., 30° C. to 80° C.). During this step, the nucleic acid primers hybridize to their respective target sequences, the isothermal polymerase initiates strand displacement and nucleic acid synthesis, and the detection reagent is, in the case of EdU, incorporated into the now-synthesizing DNA strand in place of thymidine, “tagging” every newly synthesized nucleotide molecule with an ethinyl functional group.
Step 3: Detection and Isolation. In some embodiments, the reaction mixture then undergoes a click chemistry reaction. In some embodiments, the reaction mixture is exposed to a fluorescent azide, another fluorophore, or biotin, which reacts with the EdU molecules incorporated into the newly synthesized DNA. In some embodiments, this generates a fluorescent signal within the cells comprising the genotype of interest. This allows for subsequent detection and isolation of the fluorescent cells, and further subsequent sequencing of the isolated cells.

### Example 1

The following is a non-limiting example of the present invention. It is to be understood that said example is not intended to limit the present invention in any way. Equivalents or substitutes are within the scope of the present invention.

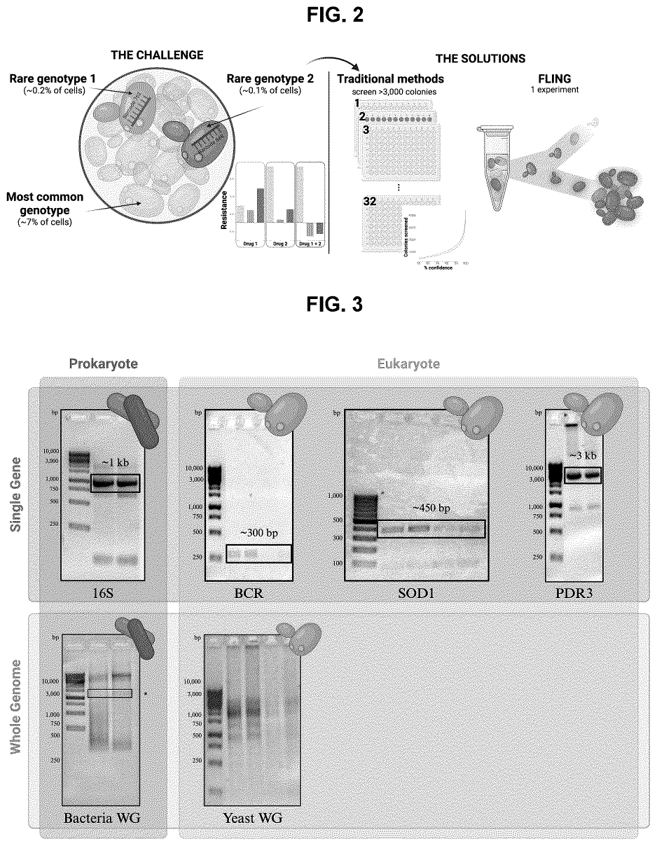

The FLING method enables robust whole genome sequencing of rare cell subpopulations of interest after those cells have been fixed in formaldehyde. In FIG. 2 an example of a case where FLING could be useful is showcased. Specifically, referred to there a case where FLING could be applied to yeast cells from a barcoded evolution experiment, which would allow one to identify the mutations present in rare drug-resistant yeast strains that have a special property: they are uniquely sensitive to multi-drug treatment.

FLING demonstrates robust performance across diverse experimental conditions, including different species, fixation/permeabilization protocols, and amplification strategies. By validating the method in both eukaryotic and prokaryotic cells, as well as through targeted and genome-wide amplification, its adaptability for a wide range of biological applications is highlighted (FIG. 3-5).

Species-Specific Robustness: To evaluate the applicability of our method across taxa, we tested intracellular amplification, and most importantly, the ability to sequence DNA amplified within formaldehyde fixed cells, in the eukaryotic model organism Saccharomyces cerevisiae (yeast) and two prokaryotic species, Escherichia coli and Bacillus subtilis. Using species-specific fixation and permeabilization protocols, we successfully amplified genomic material in all three organisms (FIG. 3). For yeast, fixation with formaldehyde followed by zymolyase treatment enabled effective permeabilization, while ethanol fixation was employed for E. coli and B. subtilis. The results demonstrate that our platform is robust to differences in cell wall composition and protocol requirements, enabling its use across phylogenetically distant organisms.

Gene-Specific Amplification: Targeted amplification and sequencing of specific genomic regions was tested, including the SOD1 and PDR3 genes, as well as an intracellular barcode region in yeast, and the 16S ribosomal RNA gene in bacteria. DNA amplified from in situ reactions was bound to streptavidin-coated beads and analyzed on an agarose gel. For all targeted amplifications, distinct bands of the expected sizes were observed, confirming the specificity and accuracy of the amplification process (FIG. 3). For example, amplification of the SOD1 locus in yeast yielded a sharp band at ~450 bp, while amplification of the bacterial 16S rRNA region generated bands corresponding to ~1.5 kb, highlighting the method's ability to isolate specific genomic features across different species. In all cases, DNA sequencing was successful and revealed that the amplified DNA pertained to the correct gene.

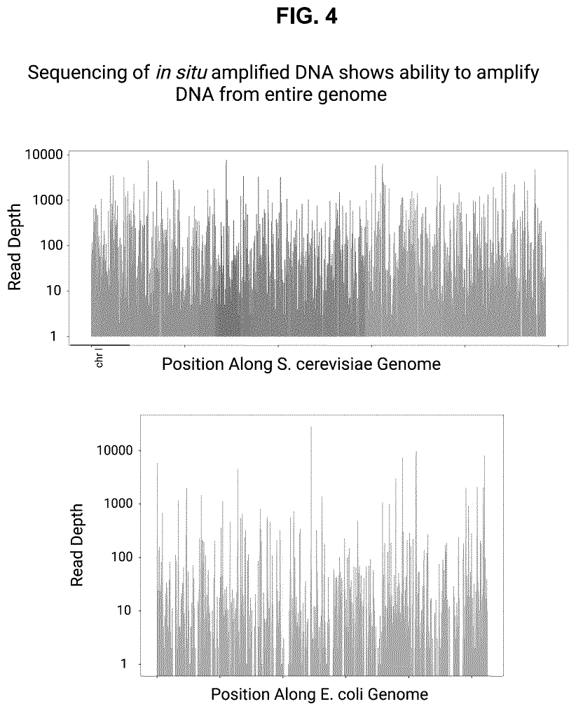

Genome-Wide Amplification: Intracellular genome-wide amplification was further tested in yeast, E. coli, and B. subtilis using random hexamers. DNA amplified from the beads was run on a gel, where smears spanning a broad range of sizes were observed, consistent with genome-wide amplification (FIG. 3). For yeast, fragment sizes ranged from 200 bp to 50 kb, while bacterial genomes yielded similar smearing patterns, reflecting uniform amplification across the entire genome. These results confirm the method's capacity for unbiased amplification of diverse genomic regions and its compatibility with organisms of varying genome sizes and complexity. Finally, and most importantly, when the DNA that was amplified in the formaldehyde fixed cells was sequenced, the entire genomes were recovered, of yeast and bacteria respectively (FIG. 4). It was further demonstrated that formaldehyde crosslinking does not limit FLING to amplifying and sequencing only short DNA fragments: the recovered DNA fragments were tens of thousands of base pairs in length, and this did not change depending on whether we fixed cells in ethanol or formaldehyde (FIG. 5).

The success of intracellular amplification across varied fixation and permeabilization protocols highlights the flexibility of the platform. For example, the use of ethanol fixation in bacteria allowed for the preservation of DNA integrity while maintaining accessibility for amplification reagents. Similarly, variations in permeabilization enzyme treatments (zymolyase for yeast, ethanol alone for bacteria) demonstrated that the method can be adapted to the structural characteristics of different cell types without compromising amplification efficiency.

Yeast cell culture. S. cerevisiae C5W4 WTC-SOD1-D102S was used. Cells were streaked on YP plus 2% dextrose agar plates from frozen −80° C. glycerol stocks and grown at 30° C. for 48 hours. Single colonies of a given yeast strain were inoculated into YP plus 2% dextrose liquid media (YPD) and grown at 30° C. with shaking [200 rotations per minute (rpm)] for 24 hours.

Bacterial cell culture. E. coli DH5a was used. Cells were streaked on LB agar plates from frozen −80° C. glycerol stocks and grown at 37° C. for 24 hours. Single colonies of DH5a were inoculated into fresh LB medium and grown at 37° C. with shaking [200 rotations per minute (rpm)] for 24 hours.

Yeast Cell Preparation and Fixation. At the time of sampling, 3 mL of yeast cultures, having been grown to saturation in YPD medium, were immediately spun down in a room-temperature centrifuge at 5000 g for 3 min. The cell pellet was fixed with 4% formaldehyde for 30 minutes at room temperature (+20° C.) and transferred to +4° C. for overnight fixation on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). The cells were centrifuged again and resuspended in 1 mL of Buffer B.

Spheroplast Formation. Cell pellets were resuspended in 0.5 ml of Spheroplasting Buffer (Buffer B containing 0.2% β-mercaptoethanol) and incubated with 2 μL of 2.5 mg/mL Zymolyase on a +4° C. block. Spheroplasting was performed at +30° C. on a rotor, and cell wall digestion was monitored by microscopy. Digestion was terminated when 90% of the cells appeared circular under the microscope, indicative of successful spheroplasting. Cells were centrifuged at 5000 g, washed with ice-cold Buffer B, and fixed in 70% ethanol at +4° C. for a minimum of 12 hours.

### Permeabilization.

Genomic DNA Amplification with EdU Labeling. To prepare the amplification reaction, cells were resuspended in the phi29 reaction mix, which contained 10× phi29 buffer, BSA (20 mg/mL), dNTPs (10 mM), sorbitol, phi29 polymerase (10,000 U/mL), primers (25 μM), and EdU (10 UM) as per Table 1. Reactions were incubated at 30° C. for 16 hours, and EdU incorporation proceeded throughout the amplification.

TABLE 1
phi29 Reaction Mix Composition

| | | |
|---|---|---|
| Reagent | Concentration | Amount per reaction |

| | | | |
|---|---|---|---|
| 10X phi29 buffer | 10X | 2.5 | μL |

| | | | | |
|---|---|---|---|---|
| BSA | 20 | mg/mL | 0.25 | μL |
| dNTPs | 10 | mM | 1.5 | μL |
| EdU | 10 | μM | 1 | μL |

| | | | |
|---|---|---|---|
| Sorbitol | 27%, 1.8M, 2M | 5.75 | μL |

| | | | | |
|---|---|---|---|---|
| phi29 polymerase | 10,000 | U/mL | 1 | μL |
| Primers | 25 | μM | 8 | μL |
| Cells | 1 | million/mL | 5 | μL |

EdU Detection. Following amplification, cells were washed twice with 3% BSA in PBS to remove excess phi29 reaction buffer. The Click-iT® reaction cocktail was prepared fresh according to Table 2 and added to each sample. Cells were incubated at room temperature in the dark for 30 minutes, washed with 3% BSA in PBS, and prepared for imaging or further analysis.

TABLE 2
Click-iT ® Reaction Cocktail Composition

| Component | Volume (per reaction) | |
|---|---|---|
| 1X Click-iT ® reaction buffer | 430 | μL |
| CuSO₄ (Component E) | 20 | μL |
| Alexa Fluor ™ azide | 1.2 | μL |
| Reaction buffer additive | 50 | μL |

The cocktail was used within 15 minutes of preparation to ensure reaction efficiency.

Flow Cytometry Analysis of EdU-Labeled Cells. After the Click-iT® EdU detection step, cells were washed twice with 1 mL of 3% BSA in PBS to remove excess reagents. The final cell pellet was resuspended in 200 μL of cold PBS and kept on ice until analysis. For flow cytometry, 100 μL of the cell suspension was loaded into an Attune NXT Flow Cytometer (Thermo Fisher Scientific). The instrument was calibrated prior to each experiment to ensure consistent performance. Fluorescence signals from Alexa Fluor™-tagged EdU-labeled DNA were measured using the appropriate laser and filter settings (e.g., excitation at 488 nm, emission at 530/30 nm for Alexa Fluor™ 488). At least 10,000 events were recorded per sample. Data were analyzed using Attune N×T software, with gating strategies applied to exclude debris and doublets and to focus on single-cell populations. Results were reported as fluorescence intensity histograms and analyzed to determine the extent of EdU incorporation in amplified DNA.

Yeast Cell Preparation and Fixation: At the time of sampling, 3 mL of yeast cultures were immediately spun down in a room-temperature centrifuge at 5000 g for 3 min. The media supernatant was removed. For formaldehyde fixation, the pellet was resuspended in 1 mL of fresh, cold, 4% formaldehyde in molecular-grade phosphate-buffered saline (PBS). After fixation, the samples were spun down in a 4° C. centrifuge at 5000 g for 3 min. The supernatant was removed, and the pellet was resuspended in 1 mL cold molecular grade 100 mM Tris HCl pH 7. For ethanol fixation, the pellet was resuspended in 1 mL of fresh, cold, 70% EtOH in molecular-grade water. After fixation, the samples were spun down in a 4° C. centrifuge at 3000 g for 5 min. The EtOH supernatant was removed, and the pellet was resuspended in 1 mL of cold molecular-grade water. For overnight fixation cells were fixed cold in a 4° C. refrigerator for approximately 18 h. For shorter fixation cells were fixed at room temperature for approximately 10 minutes. For cells fixed according to Payne et al 2021, the pellet was resuspended in 1 mL of fresh, cold, 4% formaldehyde in molecular-grade phosphate-buffered saline (PBS). Cells were fixed at room temperature for 10 min. After fixation, the samples were spun down in a 4° C. centrifuge at 5000 g for 3 min. The formaldehyde supernatant was removed, and the pellet was resuspended in 1 mL cold molecular grade PBS.

Yeast permeabilization. For permeabilization with zymolyase, after fixation cells were centrifuged and resuspended in 200 mL of 1:500 zymolyase solution [in Y Sol 1] and incubated at 37° C. for 15 minutes. Immediately after, 1 mL of cold PBS was added to the cell suspension. For permeabilization with Tween-20, samples were spun down in a 4° C. centrifuge at 5000 g for 3 min and resuspended in 250 mL of cold 0.4% Tween-20 solution [in molecular grade water] and incubated on ice for 3 min. Immediately after, 1 mL of cold PBS was added to the cell suspension. Cells were centrifuged, the supernatant was removed, and cells were resuspended in 1 mL of cold PBS. For permeabilization with TritonX-10, samples were spun down in a 4° C. centrifuge at 5000 g for 3 min and resuspended in 250 ml of cold 0.2% TritonX-10 solution [in molecular grade water] and incubated on ice for 3 min. Immediately after, 1 mL of cold PBS was added to the cell suspension. Cells were centrifuged, the supernatant was removed, and cells were resuspended in 1 mL of cold PBS. For cells permeabilized according to Payne et al 2021, samples were spun down in a 4° C. centrifuge at 5000 g for 3 min and resuspended in 250 mL of cold 0.5% TritonX-10 solution [in molecular grade water] and incubated on ice for 3 min. Immediately after, 1 mL of cold PBS was added to the cell suspension. Cells were centrifuged, the supernatant was removed, and cells were resuspended in 1 mL of cold PBS.

Bacteria fixation and permeabilization. At the time of sampling, 3 mL of bacterial cultures were immediately spun down in a room-temperature centrifuge at 5000 g for 3 min. The media supernatant was removed. For ethanol fixation, the pellet was resuspended in 1 mL of fresh, cold, 70% EtOH in molecular-grade water. Cells were fixed at room temperature for approximately 10 min. After fixation, the samples were spun down in a 4° C. centrifuge at 5000 g for 3 min. Cells were then resuspended in 1 mL of cold PBS.

Intracellular Amplification. For cells fixed and permeabilized according to Payne et al. 2021, primers were hybridized by incubating cells in 100 μL of primer hybridization mix (0.5 UM concentration primers in 2× saline-sodium citrate (SSC) buffer and 30% formamide) for 2.5-3 hours at 37 C. Cells were spun down in a 4° C. centrifuge at 5000 g for 3 min and washed twice with molecular-grade PBS. Cells were added into a phi29 master mix (10× phi29 buffer, BSA, dNTPs, Sorbitol, phi29, and primers) and incubated at 30 C for 16 hours. For all other fixation and permeabilization permutations, 5 ul of cells suspended in molecular-grade PBS were added into a phi29 master mix (10× phi29 buffer, BSA, dNTPs, Sorbitol, phi29, and primers) and incubated at 30 C for 16 hours.

Cell Lysis. After amplification, the entire reaction volume was transferred into a microcentrifuge tube and 1 mL of cold PBS and 5 μL of 10% Triton X-100 was added. Samples were spun down at 4° C., 5000 g for 3 min. The supernatant was carefully aspirated off, leaving ~30 μL to avoid removing the pellet. Cells were then resuspended in 1 mL cold PBS, spun down, and resuspended in 1 mL cold PBS for a total of two washes. After washing cells, the supernatant was aspirated off and cells were resuspended in 50 μL of cold PBS, 50 μL of 2× lysis buffer [20 mM Tris (pH 8.0), 400 mM NaCl, 100 mM EDTA (pH 8.0), and 4.4% SDS], and 10 μL of proteinase K solution (20 mg/mL). Cells were incubated at 55° C. for 2 hours with periodic vortexing to lyse the cells and reverse the formaldehyde cross-links.

Preparing Streptavidin Beads for Sample Binding. To prepare beads for sample binding, they must first be washed. For each lysate, 44 μL of Dynabeads MyOne Streptavidin C1 (Invitrogen) were washed three times with 800 μL of a 1× wash solution of 5 mM Tris-HCl pH 8.0, 1 M NaCl, 500 UM EDTA, and 0.05% Tween-20 using a magnetic 1.5 mL tube rack. The beads were then resuspended in 100 μL per sample of a 2× wash solution containing 10 mM Tris-HCl PH 8.0, 2 M NaCl, and 1 mM EDTA.

Sample Binding to Streptavidin Beads. Directly after lysates were removed from heat, 5 μL of 100 UM PMSF (resuspended in isopropanol) was added to each tube and incubated at room temperature for 10 minutes to inactivate the proteinase K. To bind DNA to C1 beads, 100 μL of resuspended C1 beads were added to each sample tube and agitated at room temperature for 60 minutes. The samples were then placed on the magnetic rack and the supernatant was removed. Samples were removed from the magnetic rack and resuspended in 250 μL of the 1× wash solution and agitated at room temperature for 5 min. Samples were replaced on the magnetic rack and the subsequent steps were repeated for a total of two wash steps. After the two 1× wash steps, the supernatant was removed, and the samples were resuspended in 250 μL of 10 mM Tris-HCl pH 8.0 and 0.1% Tween-20. At this point, the beads could be rinsed with 250 μl of molecular-grade water while the beads were still bound to the magnetic rack and moved on to subsequent steps or resuspended in 250 μL of the Tris-HCL Tween-20 buffer and stored at 4° C. overnight.

Off-the-beads Amplification. If the samples were stored overnight in the Tris-Tween buffer, the tubes were placed in a magnetic rack and the beads were rinsed with 250 μL of molecular-grade water. The bead-bound DNA was then amplified in 220 μL reactions with 2× high fidelity polymerase (KAPA HiFi) and 0.4 μM of a forward and reverse primer (which may be specific to the genotype of interest, or may comprise random hexamers) for 3 min at 95° C., and then five cycles of 98° C. for 20 s, 65° C. for 45 s, and 72 for 3 min.

Quantitative PCR. The off-the-beads PCR product was then placed against a magnetic rack and the supernatant was transferred to new optical-grade PCR tubes with qPCR dye (EvaGreen® 20×). The samples were then amplified on a qPCR machine for a further 10-20 cycles until the amplification curves exited the log-linear phase.

Size selection bead clean up. The PCR products were then cleaned using a 0.8× SPRI size selection and eluted in 20 μL of molecular-grade water.

Gel electrophoresis. 5 μL of the product which was eluted during the bead clean-up was then run on a 2% agarose gel at 120 V for 15-20 min. A single properly amplified gene should appear as a dark band on the gel at the correct size of the gene. The genome that has been properly amplified with random hexamers should appear as a smear starting at approximately 5-7 KB and ending at approximately 300 bp on a gel.

### Example 2

The following is a non-limiting example of the methods described in the present invention. It is to be understood that said example is not intended to limit the present invention in any way. Equivalents or substitutes are within the scope of the present invention.

### Fixation and Permeabilization Permutations

Fixation and permeabilization is a critical step in the method, as it preserves cellular integrity while rendering cells permeable to the amplification reagents. This balance is essential for intracellular genome amplification, where cellular structures must remain intact to contain the reaction while allowing for efficient diffusion of reagents. To this end, a range of fixation methods were explored to identify conditions that optimize these competing needs.

Formaldehyde-based fixation emerged as a versatile approach. Formaldehyde crosslinks proteins, stabilizing cellular architecture while preserving nucleic acids. Multiple protocols were tested, described below, including room-temperature fixation for short durations and overnight fixation at 4° C., a widely used method for preserving yeast cells. Both approaches demonstrated consistent results in maintaining cell morphology and enabling downstream amplification.

Ethanol fixation/permeabilization, another commonly used method, offered a complementary approach. Ethanol rapidly precipitates cellular proteins and lipids, creating a highly permeable cellular state. While less gentle than formaldehyde, ethanol fixation proved effective in permeabilizing bacterial cells such as E. coli DH5a for the following workflow, described below. This method is particularly advantageous for its simplicity and speed, as cells can be processed in under 15 minutes.

Interestingly, the choice of fixation method did not significantly impact the efficiency of genomic amplification or fluorescent labeling, highlighting the robustness of our technique. However, subtle differences in cell robustness and fluorescence intensity were noted, suggesting that FISH fixation may better preserve intracellular targets for flow cytometry applications.

Following fixation, permeabilization was required to facilitate the entry of amplification reagents. A variety of methods were tested, including enzymatic treatments with zymolyase for yeast cell walls and detergents such as Tween-20 and Triton X-100 for broader applicability. Each approach was tailored to the unique properties of the cell type and experimental needs. For yeast, zymolyase digestion of the cell wall was particularly effective, transforming the rigid cell into a spheroplast while maintaining structural integrity. In addition, detergent-based methods provided a rapid and scalable alternative for both yeast and bacterial cells.

These optimizations of fixation and permeabilization were instrumental in enabling intracellular amplification across a range of cell types. This work builds on existing protocols while adapting them for the unique demands of fluorescently labeled intracellular amplification.

Below are non-limiting examples of methods that may be used for fixation and permeabilization.
A) Overnight Formaldehyde Fixation with Zymolyase and Tween-20 Permeabilization. At the time of sampling, 3 mL of yeast cultures, grown to saturation in YPD medium, were immediately spun down in a room-temperature centrifuge at 5000 g for 3 minutes. The cell pellet was fixed with 4% formaldehyde and incubated overnight at +4° C. on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% Tween-20 at room temperature for 10 minutes before proceeding to downstream steps.
B) Overnight Formaldehyde Fixation with Zymolyase and TritonX-100 Permeabilization. At the time of sampling, 3 mL of yeast cultures, grown to saturation in YPD medium, were immediately spun down in a room-temperature centrifuge at 5000 g for 3 minutes. The cell pellet was fixed with 4% formaldehyde and incubated overnight at +4° C. on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% TritonX-100 at room temperature for 10 minutes before proceeding to downstream steps.
C) 10 Minute Formaldehyde Fixation with Zymolyase and TritonX-100 [PC1] Permeabilization (“INgen fix”). At the time of sampling, 3 mL of yeast cultures, grown to saturation in YPD medium, were immediately spun down in a room-temperature centrifuge at 5000 g for 3 minutes. The cell pellet was fixed with 4% formaldehyde for 10 minutes at room temperature (+20° C.). Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. Permeabilization was performed with 0.1% TritonX-100 at room temperature for 10 minutes before proceeding to downstream steps.
D) 30 Minute Formaldehyde Fixation with Zymolyase and Overnight Ethanol Permeabilization (“FISH fix”). At the time of sampling, 3 mL of yeast cultures, grown to saturation in YPD medium, were immediately spun down in a room-temperature centrifuge at 5000 g for 3 minutes. The cell pellet was fixed with 4% formaldehyde for 30 minutes at room temperature (+20° C.) and then transferred to +4° C. for overnight fixation on a shaker. Fixed cells were centrifuged at 5000 g for 3 minutes, the supernatant was discarded, and the pellet was resuspended in ice-cold Buffer B (1.2 M sorbitol, 0.1 M potassium phosphate dibasic, pH 7.5). Zymolyase (1 mg/mL) was added, and cells were incubated at 30° C. for 15 minutes. The permeabilization step used ethanol treatment overnight at room temperature before downstream steps.
E) Ethanol Fixation and Permeabilization. At the time of sampling, 3 mL of Escherichia coli cultures, grown to the desired density, were immediately spun down in a room-temperature centrifuge at 5000 g for 3 minutes. The cell pellet was resuspended in 70% ethanol and incubated at room temperature (+20° C.) for 10 minutes. Fixed cells were centrifuged again at 5000 g for 3 minutes, the supernatant was discarded, and the pellet was resuspended in ice-cold PBS. This washing step was repeated twice to ensure the removal of excess ethanol. Fixed cells were then processed directly for intracellular amplification or other downstream applications.

### Example 3

The following is a non-limiting example of the possible applications of the method described in the present invention. It is to be understood that said example is not intended to limit the present invention in any way. Equivalents or substitutes are within the scope of the present invention.

Targeted Amplification with Specific Primers vs. Genome-Wide Amplification with Random Hexamers: By selecting specific primers, the method enables targeted amplification of genomic loci of interest, such as genes associated with particular phenotypes or adaptive mutations. For example, using primers designed to amplify drug-resistance genes in a microbial community could help identify and isolate cells contributing to multidrug resistance. This targeted approach contrasts with genome-wide amplification using random hexamers, which is ideal for unbiased single-cell sequencing or the detection of novel mutations across the entire genome. Unlike existing methods that often require multiple separate experiments for targeted and genome-wide approaches, the platform of the present invention integrates both into a single framework, reducing time and resource expenditure.

Sorting Strategies: Pooling vs. Single-Cell Sorting: The method's compatibility with different sorting strategies allows researchers to adapt the workflow to the resolution required for their study. Pooling fluorescently labeled cells and sequencing them together is an efficient strategy for population-level studies, such as tracking the prevalence of specific genotypes in an evolving microbial community. In contrast, single-cell sorting provides the granularity needed to explore cellular heterogeneity, identify rare subpopulations, or reconstruct lineage-specific genomic changes. For instance, single-cell sorting would be essential for studies aiming to resolve heterogeneous populations within tumors or trace the genetic basis of antibiotic resistance within a bacterial population.

Selective Sequencing of Amplified DNA Using Biotinylated Primers: By incorporating biotinylated primers into the amplification process, this method can selectively enrich for newly synthesized DNA. This feature is particularly advantageous when pre-existing genomic DNA or background contamination needs to be excluded from downstream analysis. For example, biotinylated primers could be used to isolate and sequence only newly replicated DNA during studies of DNA replication dynamics or to investigate transposable element activity without interference from the original genomic template.

Applications and Customizable Workflows: Potential workflows and their corresponding applications include: a) Targeted amplification with pooled sorting for characterizing population-level diversity or detecting the presence of specific genotypes in environmental samples or microbial communities, b) Random hexamer amplification with single cell sorting for investigating rare mutations within a complex population, such as single-nucleotide polymorphisms (SNPs) or structural variations, c) Biotinylated primer-based amplification is ideal for some studies, d) A combination of FISA and genome-wide amplification could provide organism context alongside genetic information, enabling studies of mixed microbial populations where some if not all individuals are unknown or don't have a reference genome

These customizable workflows address the limitations of current methods by integrating precision, scalability, and versatility into a single platform. For example, unlike traditional fluorescence in situ hybridization (FISH), which is limited to visualizing specific loci, this method enables subsequent sequencing of the amplified DNA for detailed genetic analysis. Similarly, existing single-cell sequencing approaches often require extensive pre-amplification steps that can introduce biases or exclude low-abundance templates, challenges that this platform overcomes by combining efficient intracellular amplification with targeted or genome-wide approaches.

Overall, the versatility of this method not only broadens its applicability across diverse biological fields but also offers researchers the freedom to design workflows that align with their unique experimental goals. This adaptability positions this platform as a powerful tool for advancing genomic research in evolutionary biology, medicine, and microbial ecology.

### EMBODIMENTS

The following are non-limiting embodiments of the present invention. It is to be understood that said embodiments are not intended to limit the present invention in any way. Equivalents or substitutes are within the scope of the present invention.
Embodiment 1: A method for detecting, isolating, sequencing a genotype of interest from a mixed population of cells, the method comprising: a) fixating and permeabilizing a sample comprising the mixed population of cells; b) amplifying the genotype of interest only within the fixed and permeabilized cells possessing a genotype of interest by incubating the cells in a solution comprising at least an isothermal polymerase; c) generating a fluorescent signal within the cells comprising the genotype of interest; d) detecting and isolating the fluorescent cells; and e) sequencing the isolated cells; wherein, optionally, prior to amplifying the genotype of interest, all cells in the mixed population of cells undergo intracellular whole genome amplification using biotinylated random hexamers and isothermal polymerase, such that genetic material beyond the region used to identify the genotype of interest can be subsequently sequenced.
Embodiment 2: The method of embodiment 1, wherein the isothermal polymerase comprises a phi29 polymerase or a Bst polymerase.
Embodiment 3: The method of embodiment 1 or embodiment 2, wherein the solution further comprises enzymes, dNTPs, a buffer, and one or more nucleic acid primers.
Embodiment 4: The method of embodiment 3, wherein the nucleic acid primers comprise biotinylated primers, sequence-specific primers, or random oligonucleotide primers.
Embodiment 5: The method of any one of embodiments 1-4, wherein the solution further comprises a nucleotide analog.
Embodiment 6: The method of embodiment 5, wherein the nucleotide analog comprises 5-ethynyl-2′-deoxyuridine (EdU).
Embodiment 7: The method of embodiment 5 or embodiment 6, wherein generating the fluorescent signal comprises incubating the cells with an azide bound fluorophore.
Embodiment 8: The method of any one of embodiments 1-4, wherein generating the fluorescent signal comprises incubating the cells with a fluorescently labeled DNA probe, wherein the DNA probe has sequence homology with the genotype of interest.
Embodiment 9: The method of any one of embodiments 1-8, wherein separating the fluorescent cells comprises separating single fluorescent cells.
Embodiment 10: The method of any one of embodiments 1-9, wherein the fluorescent cells are separated by flow cytometry, microscopy, or microfluidics.
Embodiment 11: The method of embodiments 1-10 further comprising repeating the intracellular DNA amplification step on the fluorescent cells using the same or different primers.
Embodiment 12: The method of embodiments 1-10 further comprising performing additional intracellular reactions on the fluorescent cells. In some embodiments, the additional intracellular reactions comprise at least one of amplifying additional DNA, single cell RNA sequencing, or Fluorescence In Situ Hybridization (FISH).
Embodiment 13: The method of any one of embodiments 1-12 further comprising lysing the separated cells.
Embodiment 14: The method of any one of embodiments 1-13 further comprising amplifying DNA in the lysed separated cells, wherein amplifying the DNA in the separated cells comprises introducing a second solution comprising enzymes, dNTPs, a buffer, and one or more nucleic acid primers.
Embodiment 15: The method of embodiment 14, wherein the nucleic acid primers comprise biotinylated primers, sequence-specific primers, or random oligonucleotide primers.
Embodiment 16: A kit comprising: fixative agents; permeabilization agent; one or more nucleic acid primers; an isothermal polymerase; and 5-ethynyl-2′-deoxyuridine (EdU). The kit may further comprise instructions that include the methods described herein.
Embodiment 17: A kit comprising: fixative agents; permeabilization agent; one or more nucleic acid primers; an isothermal polymerase; and a fluorescent DNA probe.
Embodiment 18: The kit of embodiment 16 or embodiment 17, wherein the fixative agents comprising formaldehyde or ethanol.
Embodiment 19: The kit of any one of embodiments 16-18, wherein the permeabilization agent comprises an enzymatic treatment and/or a detergent.
Embodiment 20: The kit of embodiment 19, wherein the enzymatic treatment comprises zymolyase.
Embodiment 21: The kit of embodiment 19, wherein the detergent comprises Tween-20 or Triton X-100.
Embodiment 22: The kit of any one of embodiments 16-21, wherein the nucleic acid primers comprise biotinylated primers, sequence-specific primers, or random oligonucleotide primers.
Embodiment 23: A method for detecting, isolating, sequencing a genotype of interest from a mixed population of cells, the method comprising: fixating and permeabilizing a sample comprising the mixed population of cells; amplifying the genotype of interest within the fixed and permeabilized cells by incubating the cells in a solution comprising at least an isothermal polymerase; generating a fluorescent signal within the cells comprising the genotype of interest; detecting and isolating the fluorescent cells; and sequencing the isolated cells.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method for detecting, isolating, and sequencing a genotype of interest from a mixed population of cells, the method comprising:
a) fixating and permeabilizing a sample comprising the mixed population of cells;
b) amplifying the genotype of interest only within the fixed and permeabilized cells comprising the genotype of interest by incubating the cells in a solution comprising at least an isothermal polymerase;
c) generating a fluorescent signal within the cells comprising the genotype of interest;
d) detecting and isolating the fluorescent cells; and
e) sequencing the isolated cells.

2. The method of claim 1, further comprising, wherein, after step a), all cells in the mixed population of cells undergo intracellular whole genome amplification using biotinylated random hexamers and isothermal polymerase.

3. The method of claim 1, wherein the isothermal polymerase comprises a phi29 polymerase or a Bst polymerase.

4. The method of claim 1, wherein the solution further comprises one or a combination of enzymes, dNTPs, a buffer, and one or more nucleic acid primers.

5. The method of claim 1, wherein the solution further comprises a nucleotide analogue.

6. The method of claim 5, wherein the nucleotide analogue comprises 5-ethynyl-2′-deoxyuridine (EdU).

7. The method of claim 5, wherein generating the fluorescent signal comprises incubating the cells with the nucleotide analogue and an azide bound fluorophore, wherein the azide bound fluorophore selectively binds to the nucleotide analogue thereby generating a fluorescent signal.

8. The method of claim 1, wherein generating the fluorescent signal comprises incubating the cells with a fluorescently labeled DNA probe, wherein the DNA probe has sequence homology with the genotype of interest.

9. The method of claim 1, wherein isolating the fluorescent cells comprises separating single fluorescent cells.

10. The method of claim 1, wherein the fluorescent cells are isolated by flow cytometry, microscopy, or microfluidics.

11. The method of claim 1, further comprising, after step d), amplifying the intracellular DNA of the isolated fluorescent cells using one or more nucleic acid primers.

12. The method of claim 1, further comprising performing additional intracellular reactions on the fluorescent cells.

13. The method of claim 12, wherein the additional intracellular reactions comprise at least one of amplifying additional DNA, single cell RNA sequencing, or Fluorescence In Situ Hybridization (FISH).

14. The method of claim 1, further comprising lysing the isolated cells.

15. The method of claim 14, further comprising amplifying DNA in the lysed isolated cells, wherein amplifying the DNA in the isolated cells comprises introducing a second solution comprising enzymes, dNTPs, a buffer, and one or more nucleic acid primers.

16. The method of claim 15, wherein the nucleic acid primers comprise biotinylated primers, sequence-specific primers, or random oligonucleotide primers.

17. A kit comprising:
a) one or more fixative agent;
b) one or more permeabilization agent;
c) an isothermal polymerase;
d) a nucleotide analogue;
e) an azide bound fluorophore configured to bind the nucleotide analogue; and
f) instructions comprising a method according to claim 1.

18. The kit of claim 17, wherein the one or more fixative agent comprises formaldehyde or ethanol.

19. The kit of claim 17, wherein the one or more permeabilization agent comprises at least one of an enzymatic treatment or a detergent, wherein the enzymatic treatment comprises zymolyase, wherein the detergent comprises Tween-20 or Triton X-100.

20. The kit of claim 17, wherein the nucleic acid primers comprise at least one of biotinylated primers, sequence-specific primers, or random oligonucleotide primers.
